(19) United States
(12) Patent Application Publication
(10) Pub. No.: US 2024/0358909 A1
(43) Pub. Date: Oct. 31, 2024

(54) SURGICAL DEVICE FOR SUCTION, IRRIGATION, AND RETRACTING TISSUE

(71) Applicant: Vascular Technology, Incorporation, Nashua, NH (US)

(72) Inventors: David REGAN, Pelham, NH (US); Rachana S. SUCHDEV, Hollis, NH (US); Nilendu SRIVASTAVA, Hollis, NH (US); Kari BERCHE, Hooksett, NH (US)

(73) Assignee: Vascular Technology, Incorporation, Nashua, NH (US)

(21) Appl. No.: 18/647,906

(22) Filed: Apr. 26, 2024

Related U.S. Application Data
(60) Provisional application No. 63/498,389, filed on Apr. 26, 2023.

(51) Int. Cl. A61M 1/00; A61B 17/02

(52) U.S. Cl. CPC A61M 1/77; A61B 17/02

(57) ABSTRACT

A system for surgical suction and/or irrigation is provided that can be rendered stiff or flexible. In some embodiments, the system comprises a rod and two flexible and substantially coaligned conduits. A second conduit of the two substantially coaligned conduits can be sealed on a distal end and can have an inner diameter larger than or equal to an outer diameter of the rod. The rod can have a modulus of elasticity of at least 1 mPa. The second conduit can be configured to admit the rod while surgically inserted in a patient and without being removed from the patient. At least a first portion of a first conduit of the two substantially coaligned conduits can be bonded or coextruded to at least a second portion of the second conduit.

### TECHNICAL FIELD

This disclosure relates to surgical instruments and more particularly, to a suction and irrigation surgical system of variable rigidity.

### BACKGROUND

In surgical procedures, it is often necessary to irrigate sterile solutions into and/or aspirate bodily or irrigant fluids out of the surgical field. Traditionally, suction/irrigation devices have been designed to function as hand-held tools intended for use by the operating surgeon or an operative assistant. These hand-held devices typically incorporate a valve mechanism that the surgeon manipulates manually to control suction and irrigation functions. A common valve configuration is known as a “trumpet valve.” The trumpet valve consists of an irrigation button and a suction button, each of which can be manually depressed by the operator against a compression spring to engage a valve barrel. Manual depression of the suction button allows for aspiration through the device, while depression of the irrigation button allows for the irrigation of fluids through the device.

Recently, robot-assisted surgery has been increasingly employed by surgeons to perform technically challenging procedures in a minimally invasive fashion. In robot-assisted surgery, the operating surgeon uses a robotic console to remotely control robotic arms within the surgical field to perform the surgery. A suction/irrigation device may be manipulated by robotic arms, however doing this occupies the surgeon's attention. In addition, an assistant surgeon may be present at the patient's side to manipulate surgical tools manually, for example those tools that cannot be controlled robotically. An improvement in the field would encompass a singular suction/irrigation apparatus that could be manipulated by robotic arms or manually manipulated by an assistant at the patient's side, without removal of the apparatus from the surgical field.

### SUMMARY

In a first example, a surgical suction and irrigation apparatus is provided, the surgical suction and irrigation apparatus comprising a rod and two flexible and substantially coaligned first and second conduits wherein a second conduit of the two substantially coaligned conduits is sealed on a distal end and has an inner diameter configured to receive the rod and the first conduit is in fluid communication with a vacuum source and an irrigation source.

Example 2 includes the subject matter of Example 1, wherein the rod is at least twice as rigid as the coaligned conduits when tested using Test 1.

Example 3 includes the subject matter of Example 1, wherein the rod comprises metal, metal alloy, steel, stainless steel, glass, or a polymer.

Example 4 includes the subject matter of Example 1, wherein at least a first portion of a first conduit of the two substantially coaligned conduits is solvent bonded or coextruded to at least a second portion of the second conduit.

Example 5 includes the subject matter of Example 1, wherein the rod has a rigidity rating of A or B and the coaligned conduits have a rigidity rating of C or D by Test 1.

Example 6 includes the subject matter of Example 1, wherein the second conduit is configured to admit the rod while surgically inserted in a patient and without being removed from the patient.

Example 7 includes the subject matter of Example 1, wherein the second conduit is shorter and/or narrower than the first conduit.

Example 8 includes the subject matter of Example 1, wherein the surgical suction and irrigation apparatus is configured to be used to retract tissue of a patient while the rod is inserted in the second conduit.

Example 9 includes the subject matter of Example 1, wherein a first conduit of the two substantially coaligned conduit is configured to perform suction and/or irrigation during a surgery.

Example 10 includes the subject matter of Example 1, wherein the conduit includes a lumen therethrough and/or the distal portion of the coaligned conduits is reversibly bendable.

Example 11 includes the subject matter of Example 1, wherein the first conduit is in fluid communication with a suction/irrigation tip and the second conduit is not in fluid communication with the suction/irrigation tip.

In Example 12, a method of using a surgical suction and irrigation apparatus comprising a rod and two substantially coaligned conduits. A second conduit of the two substantially coaligned conduits can be sealed on a distal end, can have an inner diameter larger than or equal to an outer diameter of the rod, and can admit the rod. The method can comprise performing suction and/or irrigation during a surgery using a first conduit of the two substantially coaligned conduits.

Example 13 includes the subject matter of Example 12, further comprising inserting the rod into the second conduit. Performing the suction and/or irrigation can comprise performing the suction and/or irrigation using the first conduit while the rod is inserted into the second conduit. The method can further comprise removing the rod from the second conduit. Performing the suction and/or irrigation can comprise performing the suction and/or irrigation using the first conduit while the rod is not inserted into the second conduit.

Example 14 includes the subject matter of Example 13, wherein the inserting the rod and removing the rod take place while the surgical suction and irrigation apparatus is surgically inserted in a patient during the surgery.

Example 15 includes the subject matter of Example 12, wherein the rod has a modulus of elasticity of at least 1 mega Pascal.

Example 16 includes the subject matter of Example 12, wherein at least a first portion of the first conduit is solvent bonded or coextruded to at least a second portion of the second conduit.

Example 17 includes the subject matter of Example 12, wherein inserting the rod into the second conduit renders the second conduit inflexible.

Example 18 includes the subject matter of Example 1, further comprising retracting tissue of a patient using the surgical suction and irrigation apparatus while the rod is inserted in the second conduit.

In Example 19, a surgical suction and irrigation system is provided. The surgical suction and irrigation apparatus comprises a vacuum pump, an irrigation tip, a source of irrigation fluid, and two coaxial conduits comprising an inner and an outer conduit. The inner conduit has a modulus of elasticity of at least 1 megaPascal and is removable from the outer conduit of the surgical suction and irrigation apparatus.

Example 20 includes the subject matter of Example 19, wherein the outer conduit is flexible.

Example 21 includes the subject matter of Example 19, wherein the surgical suction and irrigation apparatus is configured to be used to retract tissue of a patient while the inner conduit is inserted in the outer conduit of the surgical suction and irrigation apparatus.

In Example 22, a suction and irrigation apparatus is provided. The suction and irrigation apparatus comprises a vacuum pump, an irrigation tip, a source of irrigation fluid, and two coaxial conduits comprising an inner and an outer conduit. The outer conduit is rigid or malleable. The inner conduit is removable from the outer conduit of the surgical suction and irrigation apparatus.

Example 23 includes the subject matter of Example 22, wherein the inner conduit is flexible.

Example 24 includes the subject matter of Example 22, wherein the surgical suction and irrigation apparatus is configured to be used to retract tissue of a patient while the inner conduit is inserted in the rigid or malleable outer conduit of the surgical suction and irrigation apparatus.

Example 25 includes the subject matter of Example 22, wherein the outer conduit has a rigidity rating of A or B using Test 1.

In Example 26, a method of retracting tissue at a surgical site is provided. The method comprises: inserting a flexible suction/irrigation probe into a surgical port of a patient; accessing a surgical site with the flexible suction/irrigation probe; stiffening the suction/irrigation probe while maintaining positioning of the suction/irrigation probe at the surgical site; and manipulating the stiffened suction/irrigation probe to retract tissue at the surgical site.

Example 27 includes the subject matter of Example 26, further comprising irrigating and/or suctioning the surgical site with the suction/irrigation probe.

Example 28 includes the subject matter of Example 27, further comprising irrigating and suctioning the surgical site with the suction/irrigation probe.

Example 29 includes the subject matter of Example 26, wherein stiffening the suction/irrigation probe further comprises sliding a rod through a lumen of the suction/irrigation probe.

Example 30 includes the subject matter of Example 29, wherein sliding the rod through the lumen further comprises sliding the rod through a first conduit that is adjacent to a second conduit, wherein the second conduit is in fluid communication with the surgical site.

Example 31 includes the subject matter of Example 29, wherein sliding the rod through the lumen further comprises inserting the rod in the lumen and allowing air to pass through the lumen and past the rod in a direction opposite to a direction of the insertion.

Example 32 includes the subject matter of Example 26, further comprising returning flexibility to the suction/irrigation probe after the stiffening.

Example 33 includes the subject matter of Example 32, wherein returning flexibility comprises withdrawing a rod from a lumen of a conduit.

Example 34 includes the subject matter of Example 26, wherein stiffening the suction/irrigation probe further comprises inserting a rigid conduit in the suction/irrigation probe.

Example 35 includes the subject matter of Example 34, wherein the rigid conduit comprises an inner conduit of two coaxial conduits.

Example 36 includes the subject matter of Example 34, wherein the rigid conduit comprises an outer conduit of two coaxial conduits.

In Example 37, a method of retracting tissue at a surgical site is provided. The method comprises inserting a stiff suction/irrigation probe into a surgical port of a patient, accessing a surgical site with the stiff suction/irrigation probe, rendering the suction/irrigation probe flexible while maintaining positioning of the suction/irrigation probe at the surgical site, and manipulating the suction/irrigation probe to retract tissue at the surgical site.

Example 38 includes the subject matter of Example 37, further comprising irrigating and/or suctioning the surgical site with the suction/irrigation probe.

Example 39 includes the subject matter of Example 37, wherein rendering the suction/irrigation probe flexible further comprises removing a rod from a lumen of the suction/irrigation probe.

Example 40 is a surgical suction and irrigation apparatus comprising a rigid conduit and a flexible conduit coaligned with the rigid conduit and axially slidable in relation to the rigid conduit, wherein the flexible conduit is in fluid communication with a vacuum source and an irrigation source.

Example 41 includes the subject matter of example 40 where the rigid conduit is outside the flexible conduit.

Example 42 includes the subject matter of example 40 where the flexible conduit is outside the rigid conduit.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the subject matter described herein.

### BRIEF DESCRIPTION OF THE DRAWINGS

The drawings described herein are for illustration purposes only and are not intended to limit the scope of the present disclosure in any way.

FIG. 1A illustrates example usage of a flexible suction and irrigation surgical system, in accordance with some embodiments of the disclosure.

FIG. 1B is a diagram illustrating components of a flexible suction and irrigation system, in accordance with some embodiments of the disclosure.

FIG. 1C illustrates components of a suction/irrigation system for surgery according to one embodiment.

FIG. 2 illustrates example usage of a flexible suction and irrigation device for retracting tissue during a surgery, in accordance with some embodiments of the disclosure.

FIG. 3 illustrates a perspective view of a flexible suction and irrigation surgical system having a removable rod for retracting tissue, in accordance with some embodiments of the disclosure.

FIG. 4 illustrates a perspective view of a suction and irrigation surgical system having two coaxial conduits with a stiff inner conduit for retracting tissue, in accordance with some embodiments of the disclosure.

FIG. 5a illustrates a perspective view of a suction and irrigation surgical system having two coaxial conduits with a stiff outer conduit for retracting tissue, in accordance with some embodiments of the disclosure.

FIG. 5b illustrates the example of FIG. 5a with a quick disconnect allowing the stiff outer conduit to be removed from the inner conduit.

FIG. 6 is a flow diagram of a process for using a suction and irrigation surgical system, in accordance with some embodiments of the disclosure.

FIG. 7A is a flow diagram of a process for using a suction and irrigation surgical system for retracting tissue, in accordance with some embodiments of the disclosure.

FIG. 7B is a flow diagram of a process for using a suction and irrigation surgical system for retracting tissue, in accordance with some embodiments of the disclosure.

These and other features of the present embodiments will be understood better by reading the following detailed description, taken together with the figures herein described. The accompanying drawings are not intended to be drawn to scale. In the drawings, each identical or nearly identical component that is illustrated in various figures may be represented by a like numeral. For purposes of clarity, not every component may be labeled in every drawing.

### DETAILED DESCRIPTION

As described above, robot-assisted surgery has been increasingly employed by surgeons to perform technically challenging procedures in a minimally invasive fashion. In robot-assisted surgery, the operating surgeon uses a robotic console to remotely control robotic arms within the surgical field to perform the surgery. A suction/irrigation device may be manipulated by robotic arms, however doing this occupies the surgeon's attention. In addition, an assistant surgeon may be present at the patient's side to manipulate surgical tools manually, for example those tools that cannot be controlled robotically.

As used herein, “surgical field” and “sterile field” have the meaning that they do in the art, specifically the surgical field is the space in the patient where surgery is performed. The sterile field is the area immediately around the patient where personnel and equipment need to maintain a sterile environment during surgery. The surgical field is within the sterile field.

Disclosed herein is a surgical device that provides suction/irrigation and retraction capability at the surgical site. The rigidity of the suction/irrigation probe can be altered before or during a surgical procedure. In its rigid state, the probe can be maneuvered manually by a human or robot to retract tissue or to transfer a force to objects in the surgical field. In its flexible state, the probe is easily maneuverable with graspers or other surgical tools that can grip a rigid tip at the distal end of the probe. The ability to shift between a rigid tool and a flexible one allows for the completion of more surgical tasks without requiring the changing of surgical tools and without the need to change devices or withdraw and re-insert a surgical device into the surgical field.

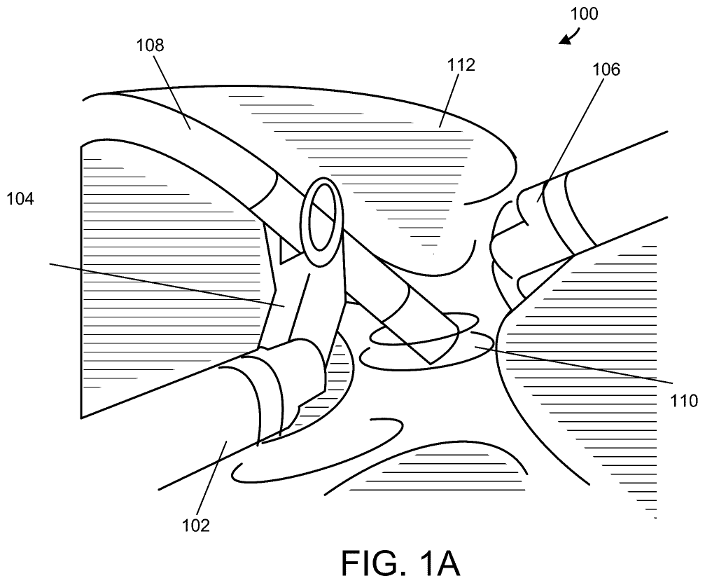

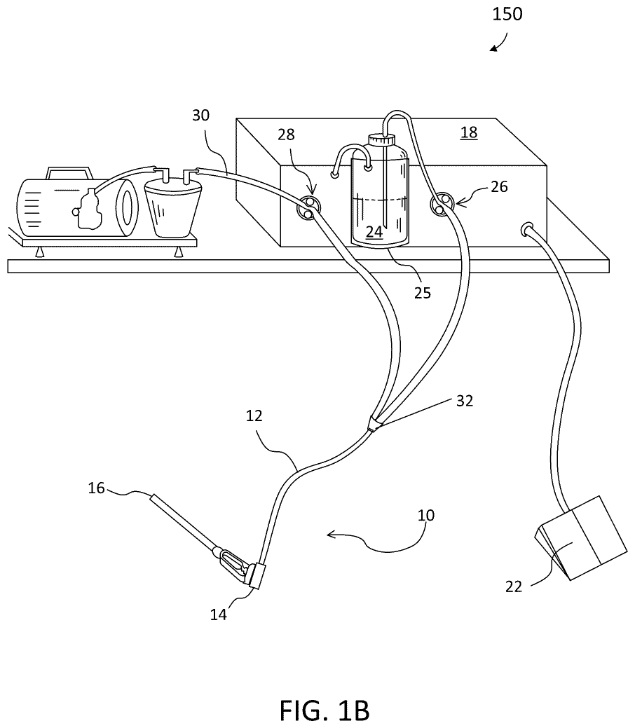

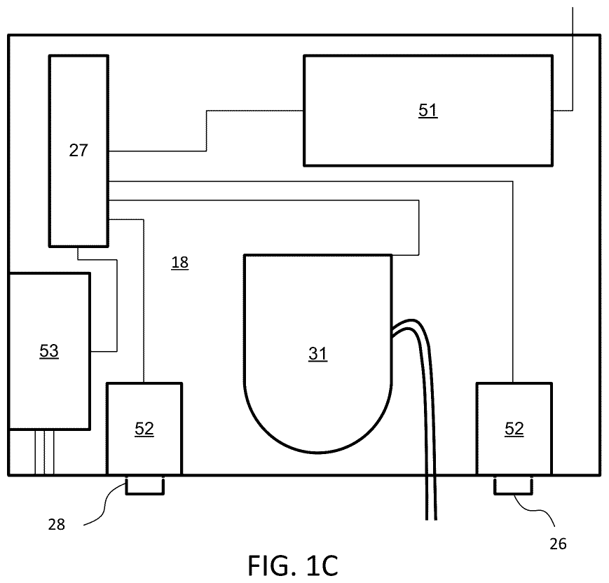

Turning now to the drawings to illustrate example embodiments of the present disclosure, FIG. 1A illustrates example usage of a flexible suction and irrigation surgical system 100, in accordance with some embodiments of the disclosure. The flexible suction and irrigation system 100 is described in greater detail in International Patent Application Nos. PCT/US2012/037516 and PCT/US2020/41658, both of which are incorporated by reference herein.

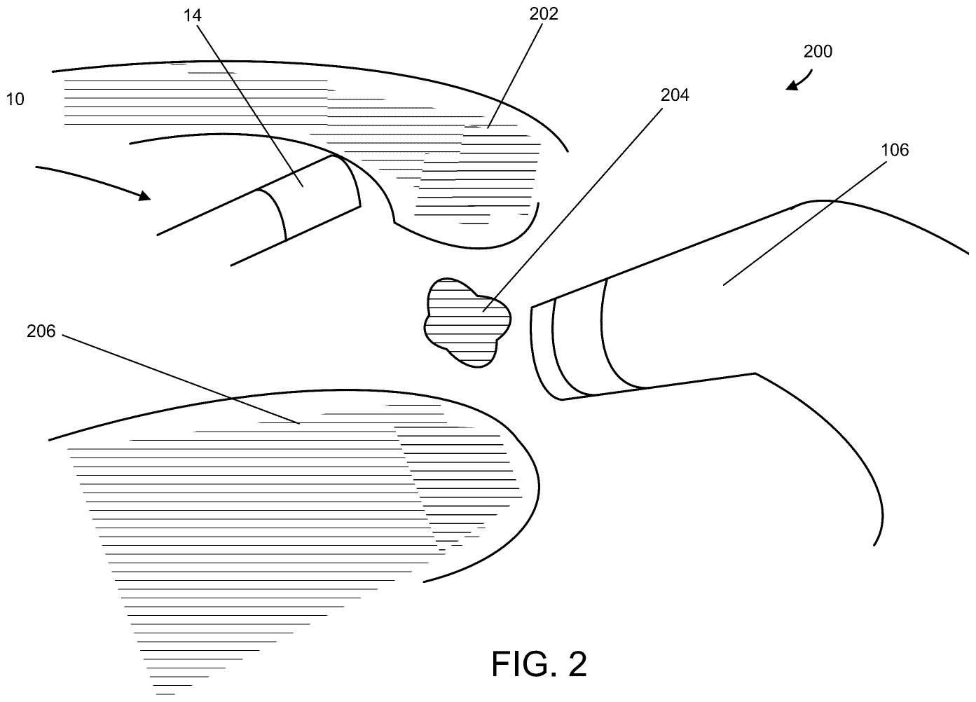

In this example, a robotic arm 102 steers graspers 104, which can manipulate a suction/irrigation probe 108 and/or the associated tubing. In this example, suction/irrigation probe 108 is being used to perform suction of fluid 110, such as to prevent pooling of blood, liquefied fat, or other bodily fluids. Suction/irrigation probe 108 can also be used to perform irrigation, such as irrigation of sterile solutions into the surgical field, and/or to irrigate such solutions out of the surgical field after their use. Alternatively, or additionally, in some cases, the suction/irrigation probe 108 can be used for insufflation, for smoke evacuation, for example to improve visibility during cauterization, and/or for tissue retraction, as in the example of FIG. 2 below. As shown, while suction/irrigation probe 108 is being used, surgical instrument 106 (for example, scissors) can simultaneously be used to perform a surgical procedure, such as on tissue 112.

As described in International Patent Application No. PCT/US2012/037516, the flexible tubing connected to suction/irrigation probe 108 can provide a number of advantages compared with inflexible suction/irrigation probes. For example, such advantages can include an increased operating range and an improved range of motion, e.g. in the surgical field. For example, the flexible design of the suction/irrigation probe 108 may allow a console surgeon to apply suction and/or irrigation 110 to areas within the surgical field that would be inaccessible with a rigid probe inserted via a fixed port. These inaccessible areas can include spaces behind membranes, tissues, organs or surgical devices that require curvature or bending of the probe to access the spaces with the probe tip. This flexible design can also provide freedom of motion at the probe tip, enabling the user to articulate the tip by 180°, such that suction or irrigation 110 can be aimed in a direction opposite to the orientation of the probe 108. Tubing materials such as braided or reinforced tubing may be used to allow for significant bending of the probe tip, without the formation of occlusive kinks in the tube lumen.

Such freedom of motion can be especially useful in minimally invasive procedures, when the suction/irrigation probe 108 and a laparoscopic or robotic camera are oriented from similar positions or angles within the surgical field. In such instances, freedom of motion at the probe tip may allow the surgeon to clean a soiled camera lens by aiming irrigation toward the camera.

FIG. 1B is a block diagram illustrating components of a flexible suction and irrigation system 150, in accordance with some embodiments of the disclosure. In various examples, the system 150 may be used to perform suction, irrigation, and/or smoke evacuation (e.g., during cauterization) so as to improve visibility.

In this example, a flexible distal suction/irrigation probe 10 comprises flexible tubing 12 and graspable tip 14 that can be inserted into a surgical field. The tubing 12 can have at least one, two or three lumen(s) and can be configured for insertion through a trocar and/or surgical port for use in minimally invasive or robot-assisted surgery. The graspable tip 14, in one simple example, may include a hollow, rigid cylindrical attachment to the tubing with suction relief holes, and may allow the surgeon to grasp the tip 14 with a surgical tool 16 and manipulate the flexible probe easily. The surgical tool 16 may comprise a tool integrated within a robotic arm (e.g., graspers 104 and/or robotic arm 102 in the example of FIG. 1A). The flexible distal probe 10 can include flexible tubing 12 and a porous, low profile, rigid, semi-rigid or flexible tip 14, which can be constructed of any biocompatible material including but not limited to polyvinyl chloride (PVC), polytetrafluoroethylene (PTFE), polyurethane, another plastic, polycarbonate, polyether ether ketone (PEEK), polyamides, nylon, silicone, and/or polyether block amide (PEBAX). Tip 14 can include a wing, as shown, to make the tip more easily graspable. Tubing 12, in various examples, may be tubing made from PVC, silicone, or C-flex type tubing, and/or any other tubing with desired biocompatibility, flexibility, and resistance to leakage or rupture even after a high number of flexure cycles.

In addition, the suction and irrigation system 150 can include a valvular unit 18 controlled through wired, wireless, or mechanical means. In one example, the user may control the valvular unit 18 with a foot pedal 22. The user input via the valvular unit 18 allows the valvular unit 18 to access a supply of sterile saline or other irrigation fluid 24 and can independently activate two valves 26 and 28. A first valve 26 can control the flow of irrigation fluid from the supply source 24, while the second valve 28 can control a line 30 running from a vacuum source such as a vacuum pump or vacuum line. As shown in FIG. 1C the system 18 may also be equipped with an internal or external power supply 51 supplying power to air pump 31 which inflates the inflation sleeve 25 to pressurize the irrigation fluid supply 24 and actuators for the valves 52. A bus bar or terminal block 27 (see FIG. 1C) can receive power from the power supply and distribute power to various components of the valvular unit 18. The system can be activated by switch 53.

The valvular unit 18 can be positioned outside of the surgical field and in or outside of the sterile field. For example, the valvular unit can be spaced from the surgical field so that it does not need to be sterile. For instance, it can be placed on a table in the same room or can be isolated in a different room. The valvular unit 18 may include valves 26 and 28 that can allow for on/off or variable flow rates. The valves can be solenoid pinch valves, which can be used to turn the suction and irrigation functions on and off. One skilled in the art will appreciate that other valves or switches may be employed, for example, mechanical solenoid valves. Power switches and a printed circuit board may be provided to allow the user to turn on the valvular unit 18 and to switch between running and setup modes. The pinch valves can be controlled via various mechanisms including (but not limited to) a foot pedal 22, additional buttons at the robotic console finger controls, buttons at and/or on the distal probe tip 12 (which may include wiring back to the control unit along the flexible tubing), voice activation or any other actuation signal communication forms.

In addition, the valvular unit irrigation fluid supply 24 may include a pumping mechanism (e.g., an air pump) to allow for “power” irrigation and/or gravity irrigation in the event of power failure. Alternatively, such a pump could be housed in a unit separate from the unit that allows for actuation of suction and/or irrigation capabilities.

As described in the example of FIG. 1A, flexible tubing 12 can provide a number of advantages compared with inflexible suction/irrigation probes, such as an increased operating range and an improved range of motion, e.g., in the surgical field. A flexible probe can be easily moved around the surgical field with a grasper operated by a human or by a robot. Such improved ranges can enable the surgeon to access surgical targets in particular areas of the surgical field without necessitating additional incisions or ports, thereby improving the safety, and reducing the complexity, of the surgery overall. For example, in a single-port surgery, a flexible suction/irrigation probe may facilitate improved angulation and manipulation within a narrow surgical field.

Nevertheless, there are at least three advantages of rigid suction/irrigation tubing. First, rigid tubing may facilitate delivering the suction/irrigation tip (e.g., tip 14) precisely to the surgical site. Second, rigid tubing may facilitate retracting (i.e., folding back) tissue or surgical devices so as to access a surgical site beneath the retracted tissue or behind a surgical device, as illustrated in the example of FIG. 2. Third, the probe can be precisely controlled by hand outside of the surgical field. Accordingly, there is a need for a suction and irrigation surgical system that can alternatively be made flexible or rigid according to the needs of the surgeon and/or assistant surgeon at the time. The disclosed system and methods can address this need.

To evaluate the rigidity of a conduit, rod or other component, the following test can be run:
Test 1: A sample of the material to be tested is anchored at one end and extended horizontally for a distance of 25 mm. A vertical force is applied in the downward direction at a point 19 mm from the anchor point. The force (Gf) required to deflect the sample by 12 mm in the vertical direction is recorded. Ratings are provided below in Table 1.

TABLE 1

| Rigidity Rating | Force (grams of force) |
|---|---|
| A | >500 |
| B | 200-500 |
| C | 100-200 |
| D | <100 |

The relative flexibility of two components can also be evaluated using the same test. For example, component X is twice as rigid (or half as flexible) as component Y if under Test 1 the force required to deflect the sample 12 mm is twice as much for component X as component Y. Component X is 50% more rigid than component Y if the force required to deflect component X a distance of 12 mm is 50% greater than the force required to deflect component Y by the same 12 mm.

In various embodiments, a rigid component can have a rigidity rating of 25% more, 50% more, 100% more or more than 500% more than the rigidity rating of a flexible component of the apparatus. In specific embodiments, a flexible conduit can have a rigidity rating of D or C. In other embodiments, a rigid component, such as a rod or rigid conduit, can have a rigidity rating of A or B.

In some embodiments, a rigid conduit or rod can be reversibly bendable. A reversibly bendable material can be bent into a different shape and then returned back to its original (or close to original) shape without breaking. A conduit or rod is reversibly bendable if a 10 cm length of it can be bent by hand by 90 degrees and can hold that shape when suspended in a horizontal plane by one end of the 10 cm length. It can also be subsequently bent to a rigid second shape, or back to its rigid original shape. An example of a reversibly bendable rod is one comprised of a malleable metal core with a polymer coating. The polymer coating can be flexible, but the malleable metal core holds its shape when bent by hand. Thus, the coated rod is a reversibly bendable material. A conduit can be reversibly bendable when its curvature is defined by the curvature of a reversibly bendable rod or second conduit that is attached to the conduit.

FIG. 2 illustrates example usage 200 of a flexible suction and irrigation device for retracting tissue 202 during a surgery, in accordance with some embodiments of the disclosure. In this example, the surgical target 204 is located behind tissue 202, and between tissue 202 and tissue 206. Accordingly, as shown, the suction/irrigation probe tip 14 is being used to retract, or pull back, tissue 202, thereby making target 204 accessible. As a result, a laparoscopic or robotic camera and/or the surgeon may have an improved view of the target 204. Moreover, the surgical instrument 106 (e.g., scissors) can now reach target 204 (for example, to cut or remove target 204). Note that, while being used to retract tissue, the probe 10 may simultaneously be operated to suction and/or irrigate fluids.

When the suction/irrigation tubing 12 is flexible, the probe 10 is steered by graspers, such as the graspers 104 and robotic arm 102 of the example of FIG. 1A and/or surgical tool 16 of the example of FIG. 1B. In particular, the flexible tubing may be difficult to guide or steer manually by the surgical assistant at the patient bedside. Moreover, due to its flexibility, the tubing may not impart leverage that would enable one to accurately and precisely steer the probe tip manually. Accordingly, as illustrated in FIG. 1A, the surgeon(s) can manipulate the flexible suction/irrigation system by grasping probe 10 with graspers either directly or via a robotic arm, for example to perform tissue retraction 200.

In some instances, the flexibility of the suction/irrigation tubing 12 may cause difficulties for retracting tissue of large size, or for placement of the suction/irrigation probe, particularly when the surgeon wishes the assistant surgeon or bedside assistant to perform these activities. For example, if the surgeon (for example, a surgeon using a console to conduct a robotic surgery) does not wish to perform the tissue retraction 200, the assistant surgeon or bedside assistant must grasp the flexible probe 10 at its proximal end as it exits the surgical port. Because of the length of the flexible tubing 12, which typically is 30 cm to 60 cm in length, the surgical assistant at the bedside may be unable to adequately and precisely control the suction/irrigation probe tip 14.

For example, consider a case where significant bleeding occurs during a surgical procedure while the suction/irrigation system is in use. In such a case, a surgeon may not wish to continue to manipulate the suction/irrigation probe to perform the suctioning. Instead, the surgeon may wish for a second party to help perform suctioning so that the surgeon can focus attention more fully on controlling the bleeding. However, the assistant surgeon or bedside assistant may not have adequate or precise control over a flexible suction/irrigation probe as outlined above.

By contrast, if the tubing can be made rigid on demand (e.g., by inserting a rod or by the other methods disclosed herein below), the assistant surgeon can manage to position the suction/irrigation probe, and/or use it to retract tissue, and the like, manually, without first securing tip 14 with graspers. In another example, if the surgeon (e.g., a surgeon conducting robotic surgery via a console) does not wish to manipulate the suction/irrigation probe to retract tissue, converting the suction/irrigation probe to have rigid tubing enables the assistant surgeon or bedside assistant to take control of the suction/irrigation probe 10, maneuver probe 10, and use it to retract tissue 202.

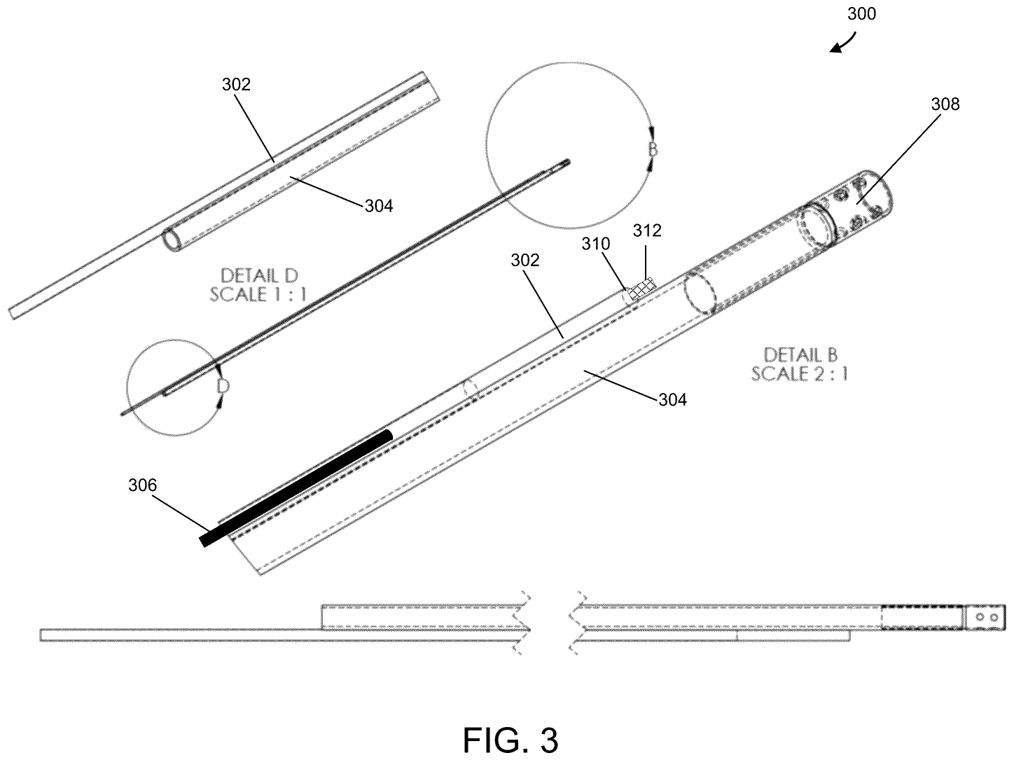

Accordingly, there is a need for a suction and irrigation surgical system that can be made alternatively flexible or rigid according to the needs of the surgeon and/or assistant surgeon at the time. In particular, there is a need for a suction and irrigation surgical system that can be made flexible or rigid while inserted into the patient, in the surgical field, without being removed. The system and methods disclosed in the examples of FIGS. 3-6 and 7A-7B below can address these needs.

FIG. 3 illustrates a perspective view of a flexible suction and irrigation surgical system 300 having a removable rod, in accordance with some embodiments of the disclosure. The flexible suction and irrigation surgical system 300 can include two adjacent flexible conduits or tubes 302 and 304. In some cases, suction and irrigation surgical system 300 may be referred to as a “spine” configuration or structure.

The tube 304 can be used for suction and/or irrigation during a surgery, as described above in the examples of FIGS. 1A-1B, and in International Patent Application No. PCT/US2012/037516. As in the example of FIG. 1B, the conduit 304 can be comprised of, or consist of, PVC, silicone, C-flex type tubing, and/or any other tubing, such as flexible tubing, and can have at least one lumen. The conduit 304 can also be fluidly connected at a distal end to a suction/irrigation tip 308 (e.g., the tip 14 of the example of FIG. 1B), and at a proximal end to a vacuum source, for example a vacuum pump or vacuum line, and/or to an irrigation fluid supply (e.g., the fluid supply 24 of the example of FIG. 1B) or to both an irrigation source and a vacuum line. The tip can be permanently rigid while the conduit portion can alternate between rigid and flexible.

Tube 302 may be substantially coaligned with tube 304 and can admit a rod 306 for the purpose of stiffening the flexible suction and irrigation surgical system 300. Tube 302 can be referred to as the rigidity control tube. Tube 304 can be referred to as the fluid flow tube. Rod 306 can be reversibly bendable. Rod 306 may have a modulus of elasticity corresponding to a stiff material, for example rod 306 may have a modulus of elasticity of at least 1 mega Pascal (MPa), at least 10 MPa, at least 100 MPa, at least 1 giga Pascal (GPa), at least 10 GPa, or at least 100 GPa. Alternatively or additionally, rod 306 may have a bending stiffness, such as a bending stiffness of at least 100 Newtons per centimeter. For example, the rod 306 may comprise steel, stainless steel, aluminum, another metal, a metal alloy, glass, or a polymer. In some cases, the rod 306 may also be referred to as a “spine.” The rod can be cylindrical and can have a diameter in the range of, for example, 1-5 mm, 0.5 to 3 mm or 1 to 3 mm. The rod can be solid or hollow and can include a groove or other negative feature along the outer surface to provide a passageway to allow air to pass when the rod is inserted or removed from closed tube 302. Alternatively, the outer surface of rod 306 can have a different profile than an inner surface of tube 302, allowing air to pass into and out of tube 302 when rod 306 is extended into, or retracted out of, the lumen of tube 302.

When rod 306 is inserted in tube 302, it can render the suction and irrigation surgical system 300 as a whole rigid or inflexible. For example, because tubes 302 and 304 can be attached or joined together, the presence of rod 306 in tube 302 can also render tube 304 and/or system 300 as a whole inflexible in practice. Note that in some embodiments the physical properties of fluid flow tube 304 are not changed but rather fluid flow tube 304 is restrained in a rigid position due to its mechanical attachment to rigidity control tube 302. Accordingly, when made inflexible by the insertion of rod 306, the probe 308 of suction and irrigation surgical system 300 can more readily be delivered for use at the surgical site, or more readily be used to retract tissue, as in the example of FIG. 2. Insertion of the rod also provides a broader area of solid contact with which the surgeon can retract. The operator can more efficiently transfer lateral force to probe 308 because of the improved stiffness of the formerly flexible tube 304. Conversely, when the rod 306 is removed from tube 302, the suction and irrigation surgical system 300 can revert to being flexible, thereby providing an increased operating range and/or improved range of motion, e.g. in the surgical field. The probe can be converted from flexible to rigid, and back again, once, twice, three or more times during a single surgical procedure. Note that the rod 306 can be inserted partially to intermediate positions along the length of conduit 302. In this way, rigidity can be provided to a portion of the probe while the distal portion retains some flexibility along, for example, one cm, two cm or five cm of the distal portion measured from the distal end. Rod 306 can be moved axially by hand or mechanically.

Tubes 302 and 304 may each have a lumen, and tube 302 may have an inner diameter larger than or equal to an outer diameter of the rod 306. In some examples, the tube 302 may be sealed on a distal end with seal 310, so as to prevent fluids in the surgical field from entering tube 302. Seal 310 can be molded as part of tube 302 or can be added. In addition, seal 310 can include a grasping tab 312 giving the surgeon another point to grasp and maneuver the apparatus. In other embodiments, a grasping tab can be attached to tube 302 at other locations on the tube. Tube 302 is not utilized for suction or irrigation, and thus may not need to connect to a suction/irrigation tip, and accordingly its distal end can be sealed, isolating rod 306 from the surgical site. In particular, the conduit 304 may be in fluid communication with a suction/irrigation tip 308, whereas the conduit 302 may not be. Likewise, the conduit 302 may be shorter than the conduit 304, since conduit 302 does not need to extend to suction/irrigation tip 308 or to valvular unit 18. For example, the conduit 302 may be several centimeters shorter than the conduit 304, and/or may be approximately the same length as the rod 306. Alternatively, conduit 302 may be the same length as conduit 304 and is not limited by the present disclosure. Whereas the proximal end of the tube 304 can be attached to a vacuum source, for example a vacuum pump or vacuum line, and/or to irrigation fluid supply 24, the proximal end of tube 302 only needs to be open and accessible for insertion or removal of the rod 306. Tube 302 need not be in fluid communication with the surgical site, the suction/irrigation tip 308, the vacuum source or the irrigation source.

In some examples, the rod 306 can be a reversibly bendable rod or tube, made of, for example, aluminum with a coating such as PTFE. In some cases, inserting a reversibly bendable rod within conduit 302 provides rigidity, while still allowing the suction and irrigation surgical system 300 to be bent to a preferred position, such as to enable working behind tissue that would not otherwise be accessible. By using a malleable material, a specific shape or curve can be imposed on tube 304 while still rendering it rigid and maneuverable. Rod 306 can be withdrawn from tube 302, its shape manually changed, and then re-inserted into tube 302 without withdrawing the suction/irrigation tip 308 from the surgical site. Alternatively, rod 306 can be shaped while inserted in rigidity control tube 302.

Tubes 302 and 304 can be two different tubes that are joined together or can be formed as a single unit. For instance, tubes 302 and 304 can be joined by solvent bonding or can be coextruded. In one embodiment, at least a portion of tube 304 can be solvent bonded (for example, using PVC cement) or coextruded to at least a portion of tube 302. For example, tubes 302 and 304 can be coextruded using two extrusion machines side by side, with tubes 302 and 304 contacting before water flows through the extrusion machines, thereby bonding the tubes together. Alternatively or additionally, tubes 302 and 304 can be joined by clips, clamps, a flexible external tube that surrounds and contains both of tubes 302 and 304 or other mechanical or physical means.

In some examples, tube 302 is configured to admit the rod and/or to have the rod removed while the suction/irrigation tip 308 is inserted in the patient. While inserting or removing the rod, the surgeon or assistant surgeon may be able to monitor the tubes via camera. For example, the surgeon or assistant surgeon may monitor the tubes via camera to prevent excessive lateral motion of the tubes while inserting the rod. To aid in monitoring rod 306, tube 302 can be comprised of material that is transparent or translucent.

Note that in some examples the separate conduit 302 keeps the rod 306 out of the path of blood or other fluids through flexible conduit 304, thereby preventing coagulation or clotting of blood. For similar reasons, the conduit 302 may be sealed on a distal end to keep blood or other fluids out and to keep air from being expelled in the surgical field. In addition, the usage of the separate conduit 302 allows insertion or removal of rod 306 from the open, proximal end of conduit 302, by contrast with the proximal end of conduit 304, which can be attached to a vacuum source and/or an irrigation fluid supply pump. The proximal end of conduit 302 can be external to the patient for access by the surgeon or assistant, and therefore may not be susceptible to the entry of blood or other bodily or surgical fluids.

In many cases, surgeries may be performed under pressurization, and the surgical port may be sealed by a seal or valve. For example, a surgeon may inflate a patient's abdomen with a gas, in order to enlarge the surgical field and provide additional space to operate. Accordingly, in some examples, the added conduit 302 may be inserted into the same surgical port as the primary fluid flow conduit 304 via the use of an air seal. For example, an air seal may include small holes or slots, and may utilize high-speed gas flow across the opening to keep the port sealed. Such an air seal can enable the use of multiple, irregularly-shaped, and/or irregularly-sized surgical instruments (such as conduits 302 and 304), while maintaining the seal (e.g., pressure and/or concentration differentials) across the surgical port. Alternatively or additionally, the surgical port can make use of a “duck bill” style valve, which can comprise plastic or another material in a triangular shape that can open to admit an instrument (e.g., the conduits 302 and 304), and subsequently close to envelop and seal around the instrument.

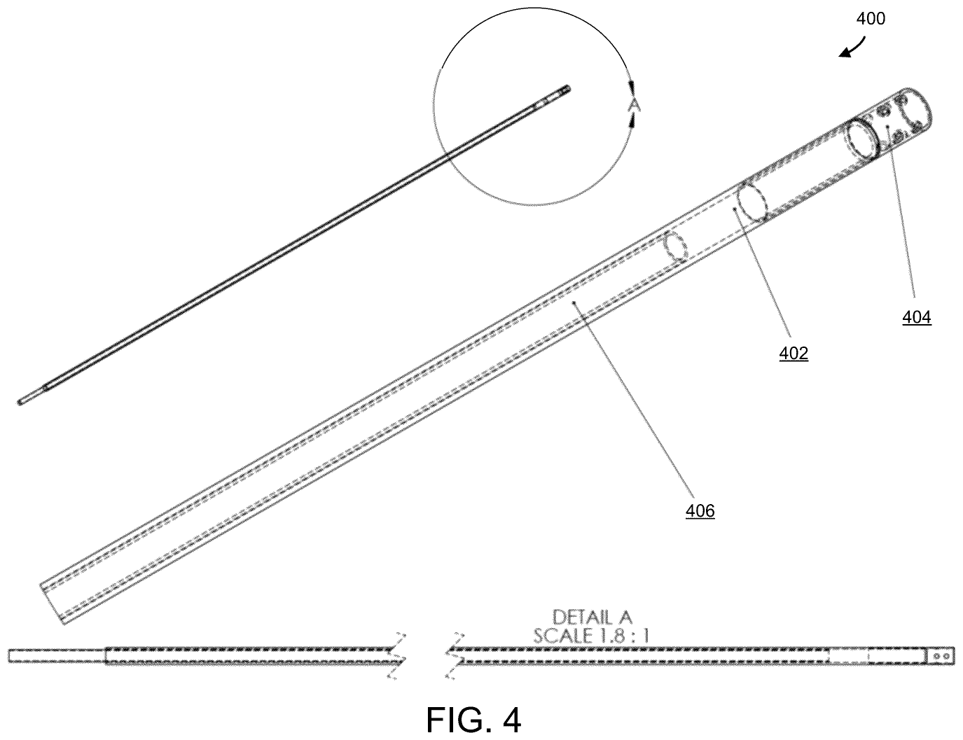

FIG. 4 illustrates a perspective view of a suction and irrigation surgical system 400 having two coaxial conduits, including a flexible outer conduit 402 and a stiff inner conduit 406, in accordance with some embodiments of the disclosure. In some cases, the suction and irrigation surgical system 400 may be referred to as an “endoskeleton” configuration or structure.

In this example, the suction and irrigation surgical system 400 includes a flexible outer conduit 402, which can be used for suction and/or irrigation during a surgery, as described in the examples of FIGS. 1A-1B above. The outer conduit 402 can be, for example, PVC, silicone, C-flex type tubing, and/or any other tubing, such as flexible tubing, and can have at least one lumen. The outer conduit 402 can be connected at a distal end to a suction/irrigation tip 404 (e.g., the tip 14 of the example of FIG. 1B), and at a proximal end to a vacuum source, for example a vacuum pump or vacuum line, and/or to an irrigation fluid supply (e.g., the supply 24 of the example of FIG. 1B).

The suction and irrigation surgical system 400 also includes a rigid inner conduit 406, which can be coaxial with the outer conduit 402, and has a smaller diameter so as to fit inside the outer conduit 402. The rigid inner conduit 406 may comprise steel, stainless steel, aluminum, another metal, glass, a polymer, or another rigid material. The rigid inner conduit 406 may have a modulus of elasticity of at least 1 MPa, at least 10 MPa, at least 100 MPa, at least 1 GPa, at least 10 GPa, or at least 100 GPa, and/or may have a bending stiffness of at least 100 Newtons per centimeter. In some examples, the primary function of rigid inner conduit 406 may be to furnish a rigid structure in suction and irrigation surgical system 400, i.e. to make suction and irrigation surgical system 400 rigid when inserted. As in the examples of FIGS. 2-3 above, making suction and irrigation surgical system 400 rigid (e.g., by inserting the inner conduit 406) can facilitate delivering the probe tip to the surgical site and can facilitate retracting tissue. In some cases, the rigid inner conduit 406 may also be referred to as an “endoskeleton.”

The rigid inner conduit 406 can also be removable from the suction and irrigation surgical system 400. Similar to suction and irrigation system 300 in the example of FIG. 3 above, suction and irrigation surgical system 400 can therefore provide the advantages of a rigid suction and irrigation probe (such as facilitating probe delivery, tissue retraction and manual manipulation by a bedside assistant) when inner conduit 406 is inserted, and the advantages of a flexible suction and irrigation probe (such as improved operating range and range of motion, and improved angulation and manipulation within the surgical field) when inner conduit 406 is removed.

In some examples, the suction and irrigation surgical system 400 must be removed from a patient in order to insert and/or remove inner conduit 406. For example, because the proximal end of the flexible outer conduit 402 may be connected to a vacuum source and/or to a pump, the suction/irrigation probe may be removed from the patient, so that the inner conduit 406 can be inserted into or removed from the outer conduit 402 via the distal suction/irrigation tip end. The suction/irrigation probe can then be reinserted into the patient.

In various embodiments conduit 406 may be shorter and not extend as far as conduit 402. This can maintain some flexibility at the distal end of the probe. In some examples, the inner conduit 406 can be mounted, stabilized, or “locked” into place within the outer conduit 402, so as to prevent inner conduit 406 from sliding out. For example, inner conduit 406 may be mounted or “locked” into place by tabs, grooves, magnets, a buckle or harness, or by another mechanism.

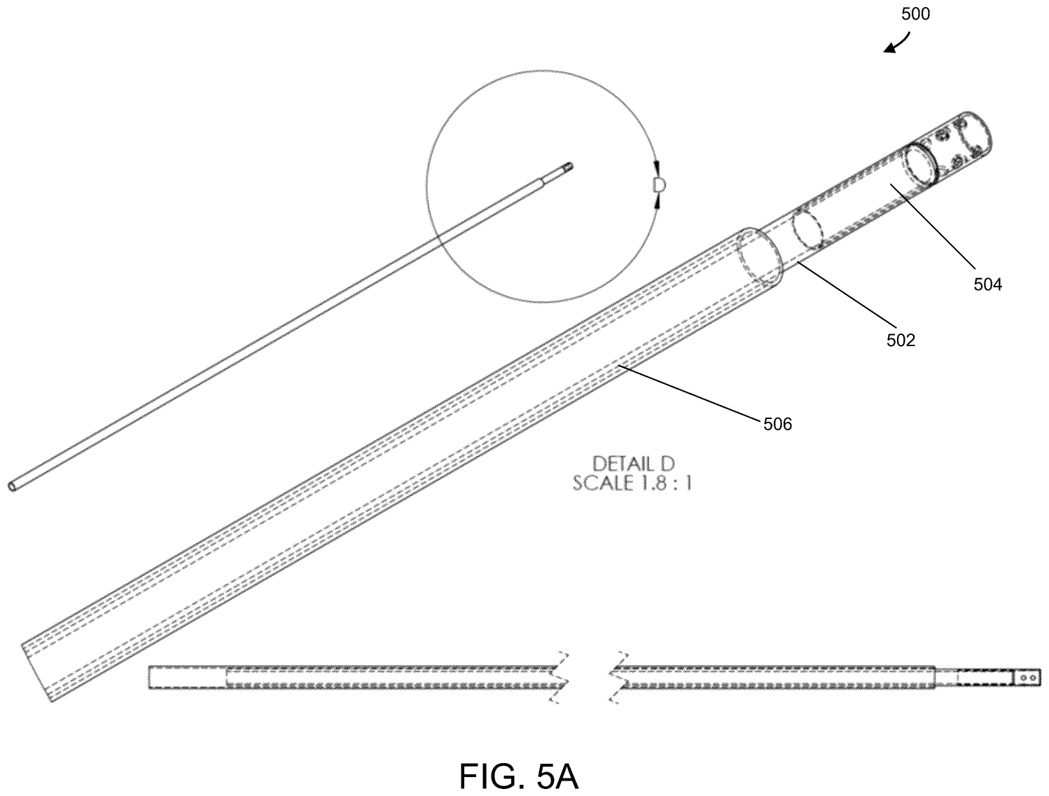

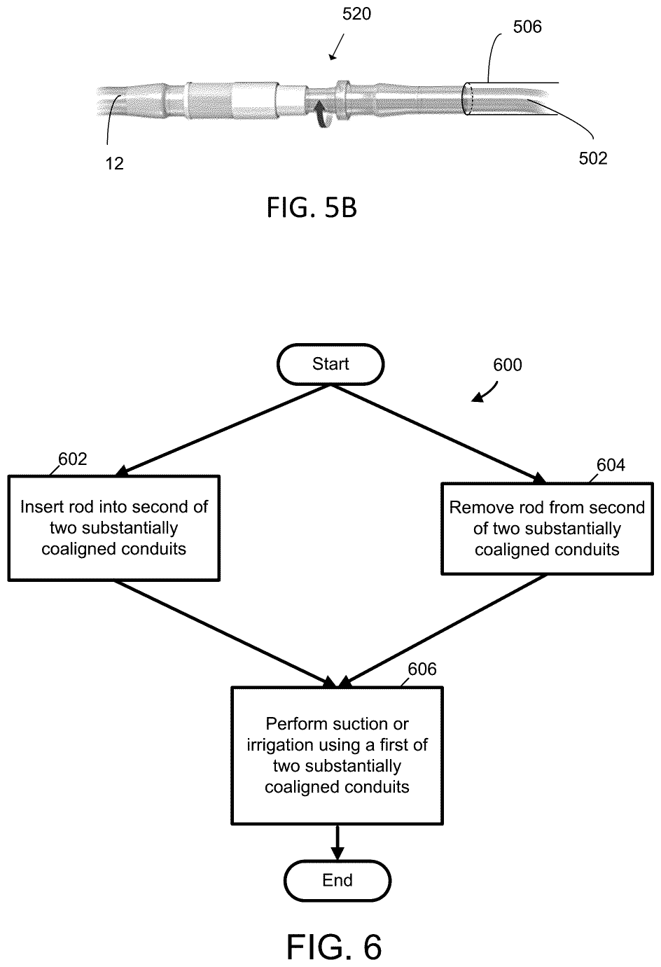

FIG. 5A illustrates a perspective view of a suction and irrigation surgical system having two coaxial conduits, including a flexible inner conduit 502 and a stiff outer conduit 506, in accordance with some embodiments of the disclosure. In some cases, the suction and irrigation surgical system 500 may be referred to as an “exoskeleton” configuration or structure.

In this example, the suction and irrigation surgical system 500 includes a flexible inner conduit 502, which can be used for suction and/or irrigation during a surgery, as described above in the examples of FIGS. 1A-1B. As in the example of FIG. 1B, the inner conduit 502 can be PVC, silicone, C-flex type tubing, and/or any other tubing, such as flexible tubing, and can have at least one lumen. The inner conduit 502 can be connected at a distal end to a suction/irrigation tip 504 (e.g., the tip 14 of the example of FIG. 1B), and at a proximal end to a vacuum source, for example a vacuum pump or vacuum line, and/or to an irrigation fluid supply pump (e.g., the pump 31 of the example of FIG. 1B).

The suction and irrigation surgical system 500 also includes a harder outer conduit 506, which can be coaxial with the inner conduit 502, and has a larger diameter so the flexible inner conduit 502 can fit inside the harder outer conduit 506. In various examples, the outer conduit 506 may be malleable or rigid. The outer conduit 506 may comprise steel, stainless steel, aluminum, another metal, glass, a polymer, or another malleable or rigid material. In some examples, the outer conduit 506 may have a coating such as Teflon. In the case of a rigid outer conduit 506, the material may have a modulus of elasticity of at least 1 MPa, at least 10 MPa, at least 100 MPa, at least 1 GPa, at least 10 GPa, or at least 100 GPa, and/or may have a bending stiffness of at least 100Newtons per centimeter. In some cases, the harder outer conduit 506 may also be referred to as an “exoskeleton.”

In some examples, the primary function of the harder outer conduit 506 may be to furnish a more rigid structure in suction and irrigation surgical system 500, i.e. to make suction and irrigation surgical system 500 as a whole relatively rigid when inner conduit 502 is inserted within outer conduit 506. As in the examples of FIGS. 2-3 above, making suction and irrigation surgical system 500 more rigid (e.g., by axially sliding the inner conduit 502 in relation to the outer conduit 506) can facilitate delivering the probe tip to the surgical site and can facilitate retracting tissue.

The rigid outer conduit 506 can also be retractable and removable from the suction and irrigation surgical system 500. Similar to suction and irrigation system 300 in the example of FIG. 3 above, suction and irrigation surgical system 500 can therefore provide the advantages of a rigid suction and irrigation probe (such as facilitating probe delivery and tissue retraction) when inner conduit 502 is inserted within harder outer conduit 506, and the advantages of a flexible suction and irrigation probe (such as improved operating range and range of motion, and improved angulation and manipulation within the surgical field) when inner conduit 502 is not housed within harder outer conduit 506.

In some examples, the suction and irrigation surgical system 500 must be removed from a patient in order to insert and/or remove outer conduit 506. For example, because the proximal end of the flexible inner conduit 502 may be connected to a vacuum source and/or to a pump, the suction/irrigation probe may be removed from the patient, so that the flexible inner conduit 502 can be inserted into or removed from the stiffer outer conduit 506 via the distal suction/irrigation tip end. The suction/irrigation probe can then be reinserted into the patient.

Alternatively, in some examples, the suction/irrigation probe 500 may be inserted into a patient via a trocar through a surgical port with the harder outer conduit 506 already mounted around the flexible inner conduit 502. In some examples, if a surgeon or assistant wishes to remove the harder outer conduit 506, the surgeon or assistant can then retract (e.g., pull back) harder outer conduit 506 as far as a Y-connector in the flexible conduit 502 (e.g., the split 32 in tubing 12 between branches 26 and 28 in the example of FIG. 1B). Accordingly, in such a case, the harder outer conduit 506 could be manually removed far enough from the inner conduit 502 so as to render the suction/irrigation probe effectively flexible, and/or could be reinserted to render the probe effectively rigid for probe delivery or tissue retraction. This can be accomplished without removing the probe from the surgical site. Alternatively, inner conduit 502 could be removably detachable at split 32, for example with the incorporation of a quick connect/disconnect inline connector (FIG. 5b, 520), allowing for the removal or insertion of conduit 506 over conduit 502 without removing conduit 502 from the surgical site. Quick disconnect 520 can be detached, the over conduit 506 can be removed, and the quick disconnect can be reattached. This can be done without removing tip 14 from the surgical field. Furthermore, outer conduit 506 can include a longitudinal slit along the length, or part of the length, of the conduit. The longitudinal slit, or cut, can allow the rigid conduit to be pried open, allowing it to be laterally removed or replaced on the flexible conduit 504 without needing to slide it off of an end of tube 504.

As described above in the example of FIG. 3, the additional outer conduit 506 may be inserted into the same surgical port normally used with only the primary fluid flow inner conduit 502, via the use of an air seal and/or a “duck bill” style valve. Such an air seal and/or “duck bill” valve can enable the use of multiple, irregularly-shaped, and/or irregularly-sized surgical instruments (such as the additional outer conduit 506), while maintaining a seal (e.g., pressure and/or concentration differentials) across the surgical port.

Any of the embodiments described above can include intermediate positions where only a portion of the conduit, near the probe, is flexible and the conduit portion proximal to the probe is stiffened. For example, instead of being fully inserted, a rod can be withdrawn 1, 2 or 3 cm to provide some flexibility to the distal portion of the conduit and probe while maintaining rigidity over the remainder of the conduit that is in the surgical site. The amount of insertion or retraction can be aided by the use of indicia on the rod (or endo or exoskeleton) that can be aligned with indicia on the irrigation/suction tube to indicate how far the end of the rod is from the probe tip.

FIG. 6 is a flow diagram of a process 600 for using a suction and irrigation surgical system, in accordance with some embodiments of the disclosure. The process 600 may be performed with a suction and/or irrigation system, such as suction and irrigation surgical system 300 of the example of FIG. 3 above.

In some examples, the process 600 can begin with inserting 602 a rod into the second of two substantially coaligned conduits. For example, the two conduits may have lumens, and may be joined by solvent bonding or coextrusion, as described in the example of FIG. 3. The first conduit may connect to a suction/irrigation tip on its distal end and to a vacuum source and/or irrigation fluid supply on its proximal end. The second conduit may be attached to the first conduit. The second conduit may be configured to admit the rod, having an inner diameter larger than or equal to an outer diameter of the rod. The second conduit may be sealed on its distal end and open on its proximal end and may be shorter than the first conduit. The second conduit may be configured to admit the rod while the second conduit is inserted in the patient. In some examples, the suction/irrigation tip may be used to access the surgical site and/or to retract tissue after the rod is inserted 602.

Alternatively, the process 600 may begin with removing 604 the rod from the second of two substantially coaligned conduits. The second conduit may be configured for the rod to be removed while the second conduit remains inserted in the patient. In some examples, the suction/irrigation tip may be used to access the surgical site and/or to retract tissue before the rod is removed 604.

Next, the process 600 may continue with performing 606 suction or irrigation using the first of the two substantially coaligned conduits. Any steps of the process can be repeated any number of times.

The process 600 may then end.

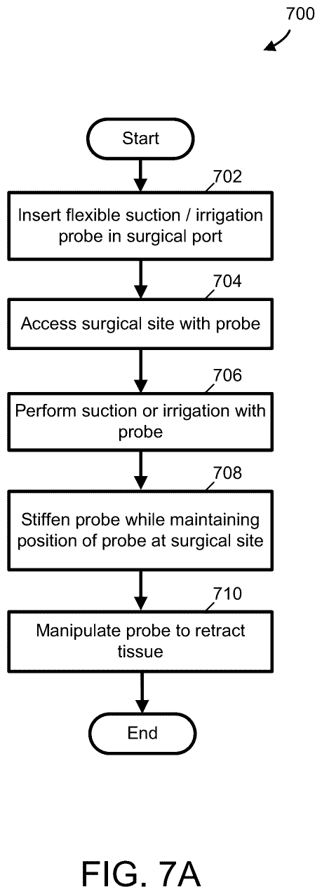

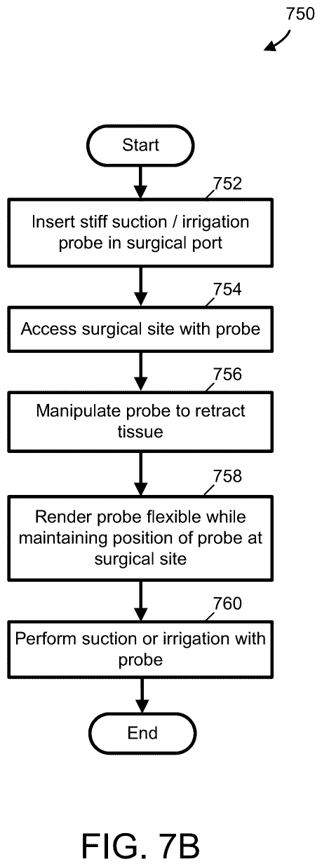

FIG. 7A is a flow diagram of a process 700 for using a suction and irrigation surgical system for retracting tissue, in accordance with some embodiments of the disclosure. The process 700 may be performed with a suction and/or irrigation system, such as suction and irrigation surgical system 300 of the example of FIG. 3 above, suction and irrigation surgical system 400 of the example of FIG. 4 above, and/or suction and irrigation surgical system 500 of the example of FIG. 5 above.

The process 700 may begin with optionally inserting 702 a flexible suction/irrigation probe into a surgical port of a patient.

Next, the process 700 may continue with optionally accessing 704 a surgical site with the flexible suction/irrigation probe.

Next, the process 700 may continue with performing 706 suction or irrigation with the flexible suction/irrigation probe. In some cases, the flexible tubing of the suction/irrigation probe can provide advantages, such as an increased operating range and an improved range of motion, e.g., in the surgical field.

Next, the process 700 may continue with stiffening 708 the suction/irrigation probe while maintaining positioning of the suction/irrigation probe at the surgical site. For example, stiffening 708 the suction/irrigation probe can include inserting a rod into one of two substantially coaligned conduits. Alternatively, or additionally, stiffening 708 the suction/irrigation probe may include inserting one of two coaxial conduits, for example an inner or outer conduit. A quick disconnect connector can allow the user to remove or add a rod without removing the probe from the surgical site.

Next, the process 700 may continue with manipulating 710 the stiffened suction/irrigation probe to retract tissue at the surgical site. In some examples, the assistant surgeon at the bedside may manually manipulate the suction/irrigation probe from outside the surgical field. Alternatively, or additionally, in some examples, the tissue retraction may be performed 710 while the suction/irrigation probe is still flexible.

The process 700 may then end.

FIG. 7B is a flow diagram of a process 750 for using a suction and irrigation surgical system for retracting tissue, in accordance with some embodiments of the disclosure. The process 750 may be performed with a suction and/or irrigation system, such as suction and irrigation surgical system 300 of the example of FIG. 3 above, suction and irrigation surgical system 400 of the example of FIG. 4 above, and/or suction and irrigation surgical system 500 of the example of FIG. 5 above.

The process 750 may begin with optionally inserting 752 a rigid or stiff suction/irrigation probe into a surgical port of a patient.

Next, the process 750 may continue with optionally accessing 754 a surgical site with the stiff suction/irrigation probe.

Next, the process 750 may continue with manipulating 756 the stiff suction/irrigation probe to retract tissue at the surgical site.

Next, the process 750 may continue with rendering 758 the suction/irrigation probe flexible while maintaining positioning of the suction/irrigation probe at the surgical site. For example, rendering 758 the suction/irrigation probe flexible can include removing a rod from one of two substantially coaligned conduits. Alternatively or additionally, rendering 758 the suction/irrigation probe flexible may include removing one of two coaxial conduits, for example an inner or outer conduit. A quick disconnect connector can allow the user to remove or add a conduit without removing the probe from the surgical site.

Next, the process 750 may continue with performing 760 suction or irrigation with the flexible suction/irrigation probe. In some cases, the flexible tubing of the suction/irrigation probe can provide advantages, such as an increased operating range and an improved range of motion, e.g. in the surgical field. Alternatively or additionally, in some examples, the suction or irrigation may be performed 760 while the suction/irrigation probe is still stiff.

The process 750 may then end.

The foregoing description has been presented for the purposes of illustration and description. It is not intended to be exhaustive or to limit the invention to the precise form disclosed. Dimensions provided are example dimensions, various alternatives are contemplated. Many modifications and variations are possible in light of this disclosure. It is intended that the scope of the invention be limited not by this detailed description. but rather by the claims appended hereto.

1. A surgical suction and irrigation apparatus comprising:
a rod; and
two flexible and substantially coaligned first and second conduits wherein a second conduit of the two substantially coaligned conduits is sealed on a distal end and has an inner diameter configured to receive the rod; and
the first conduit is in fluid communication with a vacuum source and an irrigation source.

2. The surgical suction and irrigation apparatus of claim 1, wherein the rod is at least twice as rigid as the coaligned conduits when tested using Test 1.

3. The surgical suction and irrigation apparatus of claim 2, wherein the rod comprises metal, a metal alloy, glass, or a polymer.

4. The surgical suction and irrigation apparatus of claim 1, wherein at least a first portion of the first conduit of the two substantially coaligned conduits is bonded to or coextruded to at least a second portion of the second conduit.

5. The surgical suction and irrigation apparatus of claim 1, wherein the rod has a rigidity rating of A or B and the coaligned conduits have a rigidity rating of C or D by Test 1.

6. The surgical suction and irrigation apparatus of claim 1, wherein the second conduit is configured to admit the rod while surgically inserted in a patient and without being removed from the patient.

7. The surgical suction and irrigation apparatus of claim 1, wherein the second conduit is shorter and narrower than the first conduit.

8. The surgical suction and irrigation apparatus of claim 1, wherein the surgical suction and irrigation apparatus is configured to be used to retract tissue of a patient while the rod is inserted in the second conduit.

9. The surgical suction and irrigation apparatus of claim 1, wherein the first conduit is configured to perform suction and irrigation during a surgery.

10. The surgical suction and irrigation apparatus of claim 1 wherein the rod includes an air passageway selected from at least one of a lumen through the rod and a negative feature on an external surface of the rod.

11. The surgical suction and irrigation apparatus of claim 1 wherein the first conduit is in fluid communication with a suction/irrigation tip and the second conduit is not in fluid communication with the suction/irrigation tip.

12. The surgical suction and irrigation apparatus of claim 1 wherein a distal portion of the coaligned conduits is reversibly bendable.

13. The surgical suction and irrigation apparatus of claim 1 wherein the first and second conduits do not share a common axis.

14. The surgical suction and irrigation apparatus of claim 1 wherein the second conduit includes a grasping tab.

15. A method of using a surgical suction and irrigation apparatus comprising a rod and a probe comprising two substantially coaligned flexible conduits, the method comprising:
inserting the probe into a surgical field, the probe exhibiting a first rigidity;
inserting the rod into one of the flexible conduits to increase the rigidity of the probe by at least 50%.

16. The method of using a surgical suction and irrigation apparatus of claim 15, further comprising:
performing the suction and/or irrigation using the first conduit while the rod is inserted into the second conduit; and
removing the rod from the second conduit and performing suction and/or irrigation using the first conduit while the rod is not inserted into the second conduit.

17. The method of using a surgical suction and irrigation apparatus of claim 16, wherein the inserting the rod and removing the rod take place while the surgical suction and irrigation apparatus is surgically inserted in the surgical field during surgery.

18. The method of using a surgical suction and irrigation apparatus of claim 15, the rod having a rigidity rating of A or B and the two coaligned flexible conduits have a rigidity rating of C or D.

19. The method of using a surgical suction and irrigation apparatus of claim 15, at least a first portion of the first conduit being bonded or coextruded to at least a second portion of the second conduit.

20. The method of using a surgical suction and irrigation apparatus of claim 15, wherein inserting the rod into the second conduit renders the second conduit rigid.

21-46. (canceled)
